(19) United States
(12) Patent Application Publication
(10) Pub. No.: US 2026/0183476 A1
(43) Pub. Date: Jul. 2, 2026

(54) Automated Operation of Infusion Pumps with Administrator and User Devices

(71) Applicant: Tandem Diabetes Care, Inc., San Diego, CA (US)

(72) Inventors: Anish D. Shah, Rancho Santa Margarita, CA (US); Yanni Mitis, Vista, CA (US); Andrew Stevens, Santee, CA (US)

(21) Appl. No.: 19/428,178

(22) Filed: Dec. 20, 2025

Related U.S. Application Data
(60) Provisional application No. 63/740,025, filed on Dec. 30, 2024.

(51) Int. Cl. A61M 5/142

(52) U.S. Cl. CPC A61M 5/142; A61M 2005/14208; A61M 2202/0007; A61M 2205/3303; A61M 2205/3327; A61M 2205/3576; A61M 2230/005; A61M 2230/201

(57) ABSTRACT

A system for medicament delivery may include an infusion pump and a user device both associated with a user, and an administrator device associated with an administrator of the user. The administrator device is configured to: obtain dosing information associated with the user, and generate, based on the dosing information, an instruction with respect to medicament delivery to the user. The user device is communicatively coupled to the administrator device and configured to: obtain the instruction from the administrator device, and determine a dosing request based on the instruction. The infusion pump is communicatively coupled to the user device and configured to: obtain the dosing request from the user device, and deliver a dose of medicament to the user based on the dosing request.

### RELATED APPLICATIONS

This application claims the benefit of U.S. Provisional Application No. 63/740,025 (filed Dec. 30, 2024), the entirety of which is incorporated herein by reference.

### TECHNICAL FIELD

The present disclosure relates, generally, to medical devices and, more specifically, to automated operation of infusion pumps with administrator and user devices.

### BACKGROUND

There are a wide variety of medical treatments that include the administration of a therapeutic fluid in precise, known amounts at predetermined intervals. Some devices and methods are directed to the delivery of such fluids, which may be liquids or gases. For example, ambulatory infusion pumps for delivering insulin or other medicaments can be used in conjunction with blood glucose monitoring systems, such as blood glucose meters (BGMs) and continuous glucose monitors (CGMs). Ambulatory infusion pumps typically allow a patient to adjust the amount of insulin or other medicament delivered, by a basal rate or a bolus, based on blood glucose data obtained by a BGM or a CGM, e.g., via a user device.

But in many situations, it is inconvenient or even impossible for a patient to adjust or determine the amount of medicament delivery from an infusion pump to the patient. A system based solely on user inputs from one user device, e.g., carb entries into an infusion pump, to determine a target dose is not reliable, due to limited capacity of the user device or limited capability of the user.

### SUMMARY

Embodiments of the present disclosure provide systems, apparatuses and methods for automated medicament delivery with a user device, an administrator device (e.g. a caregiver device) and an infusion pump.

In some embodiments, a system for medicament delivery includes an infusion pump and a user device both associated with a user, and a caregiver device associated with a caregiver of the user. The caregiver device is configured to: obtain dosing information associated with the user, and generate, based on the dosing information, an instruction with respect to medicament delivery to the user. The user device is communicatively coupled to the caregiver device and configured to: obtain the instruction from the caregiver device, and determine a dosing request based on the instruction. The infusion pump is communicatively coupled to the user device and configured to: obtain the dosing request from the user device, and deliver a dose of medicament to the user based on the dosing request.

In some embodiments, an infusion pump includes a processor and a non-transitory, computer-readable medium storing instructions which, when executed by the processor, cause the infusion pump to perform operations. The operations include: obtaining a dosing request from a user device associated with a user. The dosing request is determined based on an instruction with respect to medicament delivery from the infusion pump to the user. The instruction is generated by a caregiver device associated with a caregiver of the user based on dosing information associated with the user. The dosing information is received in real-time by the caregiver device. The operations further include: determining a dose of medicament for the user based on the dosing request; and delivering the dose of medicament to the user.

In some embodiments, a user device comprises a processor and a non-transitory, computer-readable medium storing instructions which, when executed by the processor, cause the user device to perform operations. The operations include: receiving an instruction from a caregiver device associated with a caregiver of a user. The user device is associated with the user. The instruction is with respect to medicament delivery from an infusion pump to the user. The instruction is generated by the caregiver device based on dosing information associated with the user. The dosing information is received in real-time by the caregiver device. The operations further include: determining a dosing request based on the instruction; and transmitting the dosing request to the infusion pump associated with the user. A dose of medicament is delivered to the user based on the dosing request.

In some embodiments, a caregiver device comprises a processor and a non-transitory, computer-readable medium storing instructions which, when executed by the processor, cause the caregiver device to perform operations. The operations include: obtaining dosing information associated with a user in real-time; generating, based on the dosing information, an instruction with respect to medicament delivery from an infusion pump to the user; and transmitting the instruction to a user device associated with the user. The caregiver device is associated with a caregiver of the user. A dosing request is determined based on the instruction. A dose of medicament is delivered to the user based on the dosing request.

In some embodiments, a computer-implemented method for operation of a caregiver device includes: obtaining dosing information associated with a user in real-time; generating, based on the dosing information, an instruction with respect to medicament delivery from an infusion pump to the user; and transmitting the instruction to a user device associated with the user. The caregiver device is associated with a caregiver of the user. A dosing request is determined based on the instruction. A dose of medicament is delivered to the user based on the dosing request.

In some embodiments, a non-transitory, computer-readable medium stores instructions which, when executed by a processor of an electronic device, cause the electronic device to perform operations. The operations include: obtaining dosing information associated with a user in real-time; generating, based on the dosing information, an instruction with respect to medicament delivery from an infusion pump to the user; and transmitting the instruction to a user device associated with the user. The caregiver device is associated with a caregiver of the user. A dosing request is determined based on the instruction. A dose of medicament is delivered to the user based on the dosing request.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention may be more completely understood in consideration of the following detailed description of various embodiments of the invention in connection with the accompanying drawings, in which:

FIG. 1 is a medical device that can be used with embodiments of the disclosure, according to various embodiments of the present disclosure.

FIG. 2 is a block diagram representing a medical device that can be used with embodiments of the disclosure, according to various embodiments of the present disclosure.

FIGS. 3A-3B depict an embodiment of a pump system, according to various embodiments of the present disclosure.

FIG. 4 is a schematic representation of a glucose monitoring system, according to various embodiments of the present disclosure.

FIG. 5 is a network environment configured for automated medicament delivery, according to various embodiments of the present disclosure.

FIG. 6 is a block diagram of a system for employing automated medicament delivery with a user device, a caregiver device and an infusion pump, according to various embodiments of the present disclosure.

FIG. 7 illustrates a scenario when one caregiver is associated with multiple users for medicament delivery, according to various embodiments of the present disclosure.

FIG. 8 illustrates a scenario when one user is associated with multiple caregivers for medicament delivery, according to various embodiments of the present disclosure.

FIG. 9 is a flow chart of a method for medicament delivery, according to various embodiments of the present disclosure.

FIG. 10 is a flow chart of a method performed by a user device for delivering a dose of medicament, according to various embodiments of the present disclosure.

FIG. 11 is a flow chart of a method performed by a caregiver device for delivering a dose of medicament, according to various embodiments of the present disclosure.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

Medical treatments may include a delivery of a therapeutic fluid in precise amounts at predetermined intervals. One category of such fluid delivery devices includes insulin injecting pumps developed for administering insulin to patients afflicted with type 1, or in some cases, type 2 diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like.

Ambulatory infusion pumps for delivering insulin or other medicaments can be used in conjunction with blood glucose monitoring systems, such as blood glucose meters (BGMs) and continuous glucose monitors (CGMs). A CGM provides a substantially continuous estimated blood glucose level through a transcutaneous sensor that estimates blood analyte levels, such as blood glucose levels, via the patient's interstitial fluid CGM systems typically consist of a transcutaneously placed sensor, a transmitter, and a monitor. Ambulatory infusion pumps typically allow a user (e.g., the patient) to adjust the amount of insulin or other medicament delivered, by a basal rate or a bolus, based on blood glucose data obtained by a BGM or a CGM, and in some cases include the capability to automatically adjust such medicament delivery. Some ambulatory infusion pumps may include the capability to interface with a BGM or CGM such as, e.g., by receiving measured or estimated blood glucose levels and automatically adjusting or prompting the user to adjust the level of medicament being administered or planned for administration or, in cases of abnormally low blood glucose readings, reducing or automatically temporarily ceasing or prompting the user temporarily to cease or reduce insulin administration. These portable pumps may incorporate a BGM or CGM within the hardware of the pump or may communicate with a dedicated BGM or CGM via wired or wireless data communication protocols, directly and/or via a device such as a smartphone.

As noted above, insulin or other medicament dosing by basal rate and/or bolus techniques could automatically be provided by a pump based on readings received into the pump from a CGM device that is, e.g., external to the portable insulin pump or integrated with the pump as a pump-CGM system in a closed-loop or semi-closed-loop fashion. With respect to insulin delivery, some systems including this feature can be referred to as artificial pancreas systems or dynamic artificial pancreas (DAP) system, because the systems serve to mimic biological functions of the pancreas for patients with diabetes. Such systems are also referred to as automated insulin delivery (AID) systems. An AID system uses measurements of metabolic signals such as interstitial glucose and user inputs such as carbohydrate entries to determine the optimal amount of insulin to deliver to maintain user blood glucose as close as possible to the euglycemic range.

When it is inconvenient or impossible for a patient to adjust or determine the amount of medicament delivery from an infusion pump to the patient, an administrator, e.g., a caregiver like a parent or healthcare professional, of the patient is often needed and relied on to monitor and/or control the medicament delivery. One objective of the present teaching is to improve the reliability of an automated medicament delivery system for delivering a medicament, e.g., insulin, to a user using an infusion pump, based on both a mobile device of the user and a mobile device of a caregiver of the user. Many computational loads and decision making processes can be shifted from the infusion pump to the two mobile devices.

In some embodiments, the infusion pump is communicatively coupled to a user device of the user, while the user device is communicatively coupled to a caregiver device of a caregiver of the user. The user device may obtain dosing information associated with the user, e.g., data from a continuous glucose monitor (CGM) associated with the user, an estimated blood-glucose level (BGL) of the user, a dosing history of the infusion pump regarding the user, or information about a current pump state of the infusion pump. The user device can transmit the dosing information in real-time to the caregiver device. As such, the caregiver is able to monitor remotely the user's health data, e.g., glucose data. Accordingly, the caregiver can generate, based on the dosing information, an instruction with respect to medicament delivery from the infusion pump to the user. In some examples, the instruction includes a bolus request for a current dosing interval regarding the medicament delivery from the infusion pump to the user. In some examples, the instruction includes an update request for updating a configuration or setting of the infusion pump. In various embodiments, the disclosed method can be applied to any infusion pump for delivering a dose of any medicament.

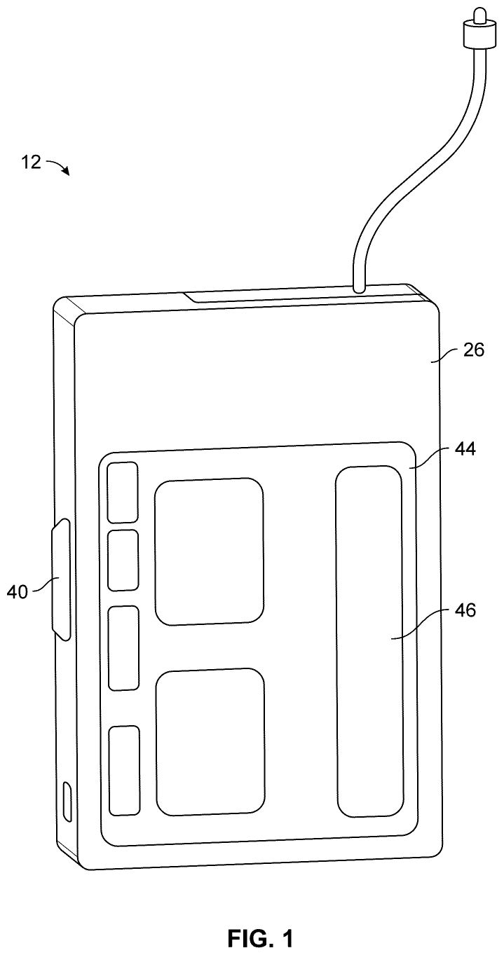

FIG. 1 depicts an embodiment of a medical device according to the disclosure. In this embodiment, the medical device is configured as a pump 12. Pump 12 may be an infusion pump that includes a pumping or delivery mechanism and reservoir for delivering medicament to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display, a remote-control device, a laptop computer, personal computer, tablet computer, a mobile communication device such as a smartphone, a wearable electronic watch or electronic health or fitness monitor, or personal digital assistant (PDA), a CGM display etc. In one embodiment, the medical device can be an ambulatory insulin pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient.

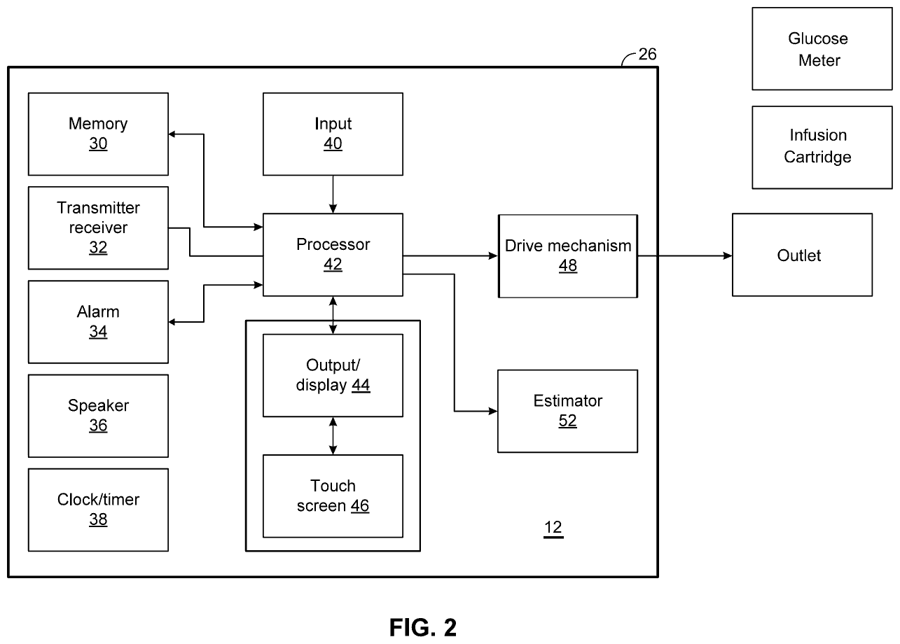

FIG. 2 illustrates a block diagram of some of the features that can be used with embodiments, including features that may be incorporated within the housing 26 of a medical device such as the pump 12 as shown in FIG. 1. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices, for example, a CGM, display device, smartphone, etc. through the transmitter/receiver. The processor 42 may also include programming that may allow the processor to receive signals and/or other data from an input device, such as a sensor that may sense pressure, temperature, or other parameters.

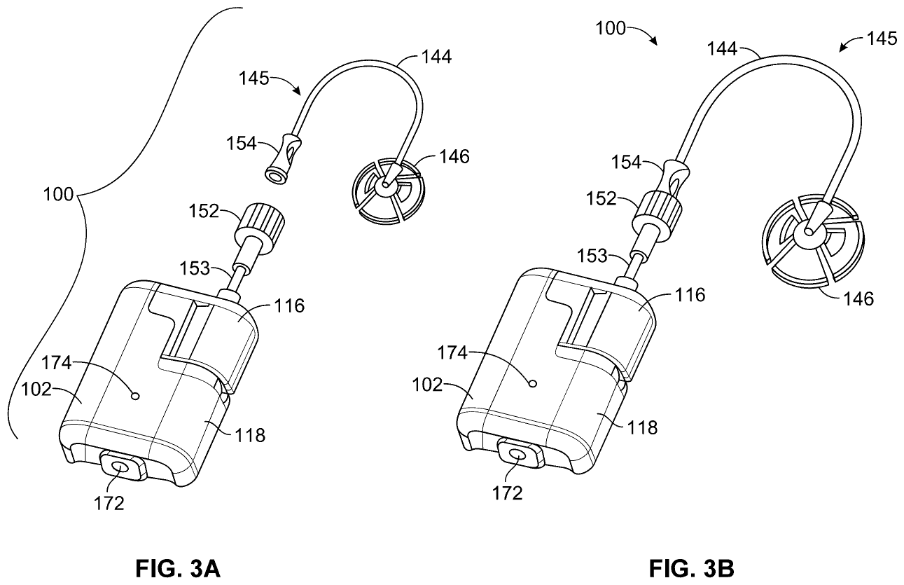

FIGS. 3A-3B depict another pump system 100 including a pump 102 that can be used with embodiments. A drive unit 118 of the pump 102 includes a drive mechanism 12 that mates with a recess in disposable cartridge 116 of the pump 102 to attach the cartridge 116 to the drive unit 118. The pump system 100 can further include an infusion set 145 having a connector 154 that connects to a connector 152 attached to pump 102 with tubing 153. A tubing 144 extends to a site connector 146 that can attach or be pre-connected to a cannula and/or infusion needle that punctures the patient's skin at the infusion site to deliver medicament from the pump 102 to the patient via infusion set 145. In some embodiments, pump can include a user input button 172 and an indicator light 174 to provide feedback to the user.

In one embodiment, the pump 102 includes a processor that controls operations of the pump and, in some embodiments, may receive commands from a separate device for control of operations of the pump. Such a separate device can include, for example, a dedicated remote control or a smartphone or other consumer electronic device executing an application configured to enable the device to transmit operating commands to the processor of the pump 102. In some embodiments, processor can also transmit information to one or more separate devices, such as information pertaining to device parameters, alarms, reminders, pump status, etc. In one embodiment, the pump 102 does not include a display but may include one or more indicator lights 174 and/or one or more input buttons 172. The pump 102 can also incorporate any or all of the features described with respect to the pump 12 in FIG. 1. Further details regarding such pumps can be found in U.S. Pat. Nos. 10,279,106 and 10,279,107 and 9,993,595, each of which is hereby incorporated herein by reference in its entirety.

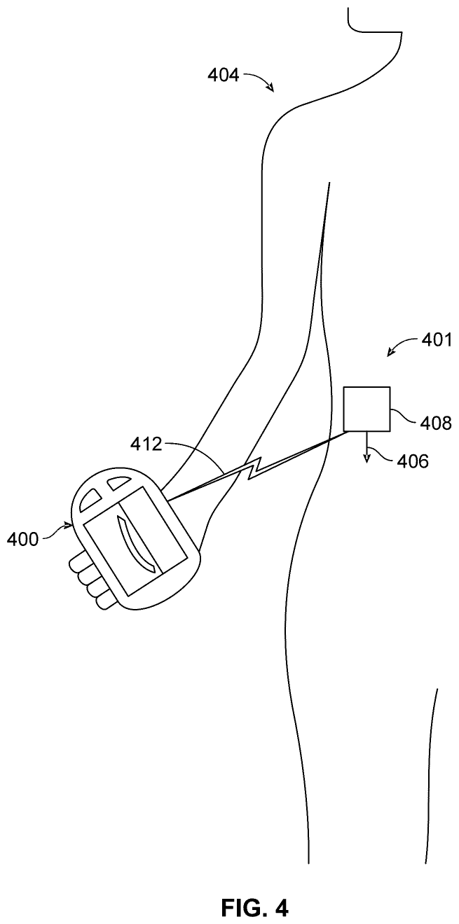

In some embodiments, the pump 12 or 102 can interface directly or indirectly (via, e.g., a smartphone or other device) with a glucose meter, such as a blood glucose meter (BGM) or a CGM. Referring to FIG. 4, an exemplary CGM system 400 according to an embodiment of the present invention is shown (other CGM systems can be used). The illustrated CGM system includes a sensor 401 affixed to a patient 404 that can be associated with the insulin infusion device in a CGM-pump system. The sensor 401 includes a sensor probe 406 configured to be inserted to a point below the dermal layer (skin) of the patient 404. The sensor probe 406 is therefore exposed to the patient's interstitial fluid or plasma beneath the skin and reacts with that interstitial fluid to produce a signal that can be associated with the patient's blood glucose level. The sensor 401 includes a sensor body 408 that transmits data associated with the interstitial fluid to which the sensor probe 406 is exposed. The data may be transmitted from the sensor 401 to the glucose monitoring system receiver 400 via a wireless transmitter, such as a near field communication (NFC) radio frequency (RF) transmitter or a transmitter operating according to a “Wi-Fi” or Bluetooth® protocol, Bluetooth® low energy protocol or the like, or the data may be transmitted via a wire connector from the sensor 401 to the monitoring system 400. Transmission of sensor data to the glucose monitoring system receiver by wireless or wired connection is represented in FIG. 4 by the arrow line 412. Further detail regarding such systems and definitions of related terms can be found in, e.g., U.S. Pat. Nos. 8,311,749, 7,711,402 and 7,497,827, as well as PCT Patent Publication No. WO 2024/119078, each of which is hereby incorporated by reference in its entirety.

In an embodiment of a pump-CGM system having a pump 12, 102 that communicates with a CGM and that integrates CGM data and pump data as described herein, the CGM can automatically transmit the glucose data to the pump. The pump can then automatically determine therapy parameters and deliver medicament based on the data. Such an automatic pump-CGM system for insulin delivery can be referred to as an automated insulin delivery (AID) or an artificial pancreas system that provides closed-loop therapy to the patient to approximate or even mimic the natural functions of a healthy pancreas. In such a system, insulin doses are calculated based on the CGM readings (that may or may not be automatically transmitted to the pump) and are automatically delivered to the patient at least in part based on the CGM reading(s). In various embodiments, doses can be delivered as automated correction boluses and/or automated increases or decreases to a basal rate. Insulin doses can also be administered based on current glucose levels and/or predicted future glucoses levels based on current and past glucose levels.

For example, if the CGM indicates that the user has a high blood glucose level or hyperglycemia, the system can automatically calculate an insulin dose necessary to reduce the user's blood glucose level below a threshold level or to a target level and automatically deliver the dose. If the CGM data indicates that the user has a low blood glucose level or hypoglycemia, the system can, for example, automatically reduce a basal rate and/or make other suggestions as may be appropriate to address the hypoglycemic condition. As with other parameters related to therapy, such thresholds and target values can be stored in memory located in the pump and the pump processor can periodically and/or continually execute instructions for a checking function that accesses these data in memory, compares them with data received from the CGM and acts accordingly to adjust therapy. The complexity of the algorithm used to calculate the insulin doses is therefore limited by capabilities of the pump processor, memory, battery, etc.

In some embodiments, systems include an infusion pump, which can receive dosing functions from an electronic device or a neural network, select one of the dosing functions based on characteristics of a user, deliver a dose of medicament to the user according to the selected dosing function. The characteristics may include a basal rate, a correction factor, or a matching factor estimated based on the basal rate and the correction factor. In some embodiments, the electronic device may be a mobile device (e.g., a phone, a tablet, a computer, etc.) of the user. The mobile device of the user (called user device) typically has more computation power than the infusion pump. For example, a smartphone can have 5 to 10 orders of magnitude more capability than an embedded device like an infusion pump. As such, various functions and algorithms for computing the insulin doses can be shifted from the pump processor to a processor at the mobile device. The infusion pump and the mobile device can be connected to each other via Internet, Wi-Fi, Bluetooth, near field communication (NFC), etc. In some embodiments, the user device of the user is further connected to a caregiver device of a caregiver, who is responsible to take care of the user regarding the medicament delivery to the user.

Patients with diabetes often rely on caregivers for support in managing their insulin delivery. One purpose of the present disclosure is to provide embodiments to enhance a caregiver's ability to manage patient therapy by enabling remote bolus delivery. That is, caregivers can remotely deliver a bolus to a patient via a secure communication flow involving e.g., a caregiver app, a cloud backend, and the patient's insulin pump (through a mobile control app). The remote bolus delivery may be made when the caregiver is alerted with therapy data that indicates a need for immediate insulin administration. This ensures timely and safe insulin delivery, by a remote bolus delivery system that allows caregivers to monitor therapy data and initiate bolus administration remotely using a secure multi-layer communication flow.

In some embodiments, a disclosed system includes: a continuous glucose monitor (CGM), an insulin pump, a mobile control app, a cloud backend, and a caregiver mobile app. The CGM can measure the patient's glucose levels and send glucose readings to the insulin pump, e.g., via Bluetooth. The insulin pump may deliver insulin to the patient, with a connection to the CGM for glucose readings and a connection to the mobile control app (e.g., via Bluetooth) for bolus requests and data upload. The mobile control app may receive bolus requests from the cloud backend, verify command authenticity and patient-specific safety limits (e.g., maximum bolus volume), and provide confirmation of bolus delivery back to the cloud backend after sending bolus requests to the insulin pump. The mobile control app can also upload therapy data (e.g., insulin events and CGM readings) to the cloud backend, and implement safety checks to ensure multiple duplicate bolus requested are not made.

In some embodiments, the cloud backend can: accept bolus requests from the caregiver app, route the requests securely to the mobile control app, log remote bolus events for audit and tracking, and implement safety checks (e.g., frequency and size of bolus) to prevent accidental overdosing. Further, the cloud backend may also store patient data (e.g. therapy and profile) and stream therapy data (e.g. insulin and CGM readings) to the caregiver app in near real-time.

In some embodiments, the caregiver mobile app may include an interface for remote bolus delivery, authenticate and authorize caregivers before sending bolus requests, and provide alerts and confirmations for bolus actions to caregivers. The caregiver mobile app allows caregivers to monitor therapy data and receive alerts, and provides notifications for abnormal glucose levels or other therapy data.

In some embodiments, a caregiver uses a caregiver mobile app to monitor glucose and insulin therapy data. Upon detecting abnormal glucose levels, the caregiver can send a bolus delivery request through the caregiver mobile app. The request is securely sent to the cloud backend, which forwards it to a mobile control app. The mobile control app issues a bolus delivery command to the insulin pump (e.g., via Bluetooth). After delivery, a confirmation of successful delivery is sent back to the caregiver. As such, the caregiver mobile app allows caregivers to send a bolus request and confirm delivery. The cloud backend routes requests securely to the patient's mobile control app, ensuring authentication, safety checks, and logging of bolus events. The mobile control app issues the bolus command to the insulin pump via Bluetooth. The insulin pump executes bolus delivery and sends feedback to confirm successful execution. In some embodiments, a patient approval may be required by the system for bolus execution an additional safety feature.

In some example, during a monitoring phase, a caregiver receives alerts from the caregiver app about abnormal glucose levels or other therapy concerns. The caregiver app may display real-time therapy data, including glucose levels and insulin activity. Then, the caregiver can select an option to deliver a bolus in the caregiver app, specify a bolus amount (within pre-defined safety limits) and confirm the request. The bolus request is then sent securely to the cloud backend, which performs authorization checks (e.g., verify caregiver permissions) and safety checks (e.g., ensure the bolus does not exceed patient-specific limits or frequency constraints). After authorization and safety checks, the backend may securely forward the bolus request to the mobile control app. After the mobile control app receives the bolus request from the backend, the mobile control app may issue a bolus delivery command to the insulin pump (e.g., via Bluetooth). The pump then executes the bolus delivery and logs the event.

In some embodiments, during a feedback loop, the insulin pump can send a confirmation of the bolus delivery back to the mobile control app. The mobile control app can send updates to the cloud backend with the delivery status. The backend then updates therapy data. The caregiver app also notifies the caregiver of successful delivery.

The caregiver in these embodiments may refer to any of: a health caregiver, a parent, a healthcare provider, etc. In some use cases of the disclosed embodiments: a caregiver may detect high glucose levels via an alert and remotely deliver a bolus to prevent hyperglycemia; a parent managing a child's therapy can intervene even when physically distant; a healthcare provider can remotely support patients during critical situations.

Some disclosed embodiments can enable an integration of remote bolus delivery with caregiver monitoring, with a secure communication flow involving a cloud backend and mobile control app to deliver insulin, and include a real-time feedback on bolus delivery to both the cloud backend and caregiver app.

In some embodiments, for security, the system ensures end-to-end encryption for all data and commands, and implements strong authentication for caregivers to prevent unauthorized bolus requests. In some embodiments, as safety mechanisms, the system can establish limits for bolus size and frequency, and may introduce overrides requiring patient consent for larger boluses, if feasible. In some embodiments, for regulatory compliance, the system can ensure adherence to medical device regulations, e.g., including FDA and ISO standards for software as a medical device (SaMD). In some embodiments, thorough testing may be conducted in simulated and real-world conditions to ensure reliability and safety.

Various embodiments of the present disclosure can improve patient safety with timely intervention by enabling caregivers to deliver a bolus promptly when the caregivers detect abnormalities, reducing the risk of hyperglycemia. The caregivers can manage therapy remotely without requiring patient or local intervention. The disclosed system can enhance monitoring by providing real-time visualization, confirmation and feedback on bolus delivery and execution and corresponding impact on therapy. By enabling remote bolus delivery, the system addresses critical therapy needs and ensures patients receive timely interventions, ultimately improving outcomes and caregiver support.

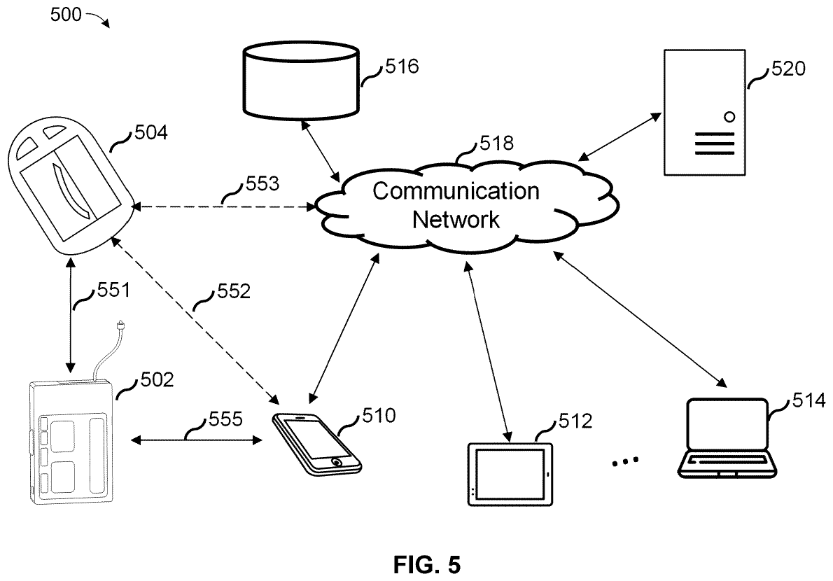

Embodiments disclosed herein employ a dynamic medicament infusion system that includes both the user device and the caregiver device, together with an infusion pump configured to deliver medicament doses to the user. FIG. 5 is a network environment 500 configured for automated medicament infusion, e.g., automated insulin delivery, according to various embodiments of the present disclosure.

As shown in FIG. 5, the network environment 500 includes a plurality of devices or systems configured to communicate over one or more network channels, illustrated as a communication network 518. For example, in various embodiments, the network environment 500 can include, but not limited to, a pump 502, a monitoring system 504 (e.g., a BGM system or a CGM system), a database 516, a server 520 and one or more computing devices 510, 512, 514 operatively coupled over the communication network 518. The pump 502, the monitoring system 504, the server 520 and the multiple computing devices 510, 512, 514 can each be any suitable computing device that includes any hardware or hardware and software combination for processing and handling information. For example, each can include one or more processors, one or more field-programmable gate arrays (FPGAs), one or more application-specific integrated circuits (ASICs), one or more state machines, digital circuitry, or any other suitable circuitry. In addition, each can transmit and receive data over the communication network 518.

In some examples, the pump 502 can be implemented as the pump 12 shown in FIG. 1 and FIG. 2, or as the pump 102 shown in FIG. 3. The monitoring system 504 may be implemented as the CGM system 400 shown in FIG. 4. In some examples, each of the multiple computing devices 510, 512, 514 can be a cellular phone, a smart phone, a tablet, a personal assistant device, a voice assistant device, a digital assistant, a laptop, a computer, or any other suitable device. In some examples, each of the multiple computing devices 510, 512, 514 includes one or more processing units, such as one or more graphical processing units (GPUs), one or more central processing units (CPUs), and/or one or more processing cores.

In some examples, the pump 502, the monitoring system 504, and the computing device 510 are all associated with a same user. The monitoring system 504 is configured to monitor blood glucose level of the user, by continuously estimating the blood glucose level at each time step. The pump 502 is configured to deliver medicament, e.g., insulin, to the user based on the estimated blood glucose level from the monitoring system 504. The computing device 510 is owned by or associated with the user. For example, an account of the user has been logged in at the computing device 510. In addition, the same account of the user is also linked to or associated with the pump 502 and the monitoring system 504.

In some examples, one of the computing devices 512, 514 is owned by or associated with a caregiver of the user. The caregiver may be any one of: parents of the user, healthcare professionals for the user, school nurses of the user, responsible adults of the user, or legal representatives of the user. For example, while the computing device 510 is a user computing device 510 associated with the user, the computing device 512 is a caregiver computing device 512 associated with the caregiver of the user. The computing device 514 may be either another user computing device associated with another user taken care of by the caregiver, or another caregiver computing device associated with another caregiver taking care of the user. In some embodiments, a software application is installed on both the user computing device 510 and the caregiver computing device 512. While a patient account is signed in by the user via the software application on the user computing device 510, a caregiver account is signed in by the caregiver via the software application on the caregiver computing device 512. Based on a previous agreement from the user, the user may have authorized the caregiver account to be associated with the patient account.

As shown in FIG. 5, the multiple computing devices 510, 512, 514 are connected to each other via the communication network 518, which may be a cloud network handled by the server 520 in association with the database 516. In some embodiments, other methods (e.g., based on Bluetooth or NFC) can be used to pair the pump 502 and/or the monitoring system 504 to the computing device 510. The pairing information may be saved securely at all these devices even when they are disconnected (e.g., due to power off or network interruption). As such, when they are reconnected via the communication network 518, they can be paired again automatically.

In some embodiments, to implement more powerful and complicated algorithms for medicament dose determination, and to avoid computational load of the algorithms at the pump 502, at least one of the multiple computing devices 510, 512, 514 works in cooperation with the pump 502 to compute optimal insulin doses at each time step for delivery to the user. For example, the user computing device 510 may be connected to the pump 502 via a direct link (e.g., Wi-Fi, Bluetooth or NFC), and obtain dosing information associated with the user from the pump 502. The dosing information may include but not limited to: data from the monitoring system 504, an estimated blood glucose level of the user, a dosing history of the pump 502 to the user, and information about a current pump state of the pump 502.

In some embodiments, the communication network 518 handled by the server 520 in association with the database 516 can enable the dosing information to be visualized through applications on devices connected to the communication network 518, e.g., on the user computing device 510 and on the caregiver computing device 512. The scheme in FIG. 5 also allows a caregiver to be able to remotely monitor a patient's health data or glucose level, and react in real-time to the monitored data by triggering a bolus or changing a setting of the pump.

The user computing device 510 can determine a dosing request based on the dosing information. In some embodiments, the user computing device 510 transmits the dosing information in real-time to the caregiver computing device 512, via the communication network 518, for the caregiver to monitor in real-time the data related to medicament delivery to the user. In some embodiments, the dosing information received by the caregiver computing device 512 may induce or trigger the caregiver computing device 512 to generate, based on the dosing information, an instruction with respect to medicament delivery from the pump 502 to the user. The instruction may include a bolus request for a current dosing interval regarding the medicament delivery and/or an update request for updating a configuration or setting of the pump 502. In some embodiments, the dosing request is determined by the user computing device 510 based on the dosing information and the instruction. After receiving the dosing request from the user computing device 510, the pump 502 may determine a dose of medicament based on the dosing request, and deliver the dose of medicament to the user.

Although FIG. 5 illustrates three computing devices 510, 512, 514, the network environment 500 can include any number of computing devices 510, 512, 514. Similarly, the network environment 500 can include any number of the pumps 502, the monitoring systems 504, the servers 520 and the databases 116. That is, multiple users'medicament infusion systems can be implemented via the communication network 518 in the network environment 500.

The communication network 518 can be a WiFi® network, a cellular network such as a 3GPP® network, a Bluetooth® network, a satellite network, a wireless local area network (LAN), Wi-Fi network, a network utilizing radio-frequency (RF) communication protocols, a Near Field Communication (NFC) network, a wireless Metropolitan Area Network (MAN) connecting multiple wireless LANs, a wide area network (WAN), or any other suitable network. The communication network 518 can provide access to, for example, the Internet.

In some embodiments, the pump 502 can access the communication network 518 via the user computing device 510. In some embodiments, a caregiver account signed in by the caregiver on the caregiver computing device 512 is authorized (e.g., based on a previous agreement from the user) to instruct a patient account signed in by the user on the user computing device 510, e.g., regarding next bolus or future setting of the pump 502.

In some embodiments, the caregiver account on the caregiver computing device 512 is authorized to instruct a plurality of patient accounts, including the patient account on the user computing device 510. For example, the same caregiver is taking care of multiple patients. The instruction transmitted from the caregiver computing device 512 to the user computing device 510 includes at least one identification uniquely identifying the patient account among the plurality of patient accounts, or uniquely identifying the user computing device 510 among a plurality of user computing devices associated with the caregiver computing device 512.

In some embodiments, the patient account on the user computing device 510 is configured to receive instructions from a plurality of authorized caregiver accounts, including the caregiver account on the caregiver computing device 512. For example, the same user is taken care by different types of caregivers including e.g.: parents of the user, healthcare professionals for the user, school nurses of the user, responsible adults of the user, legal representatives of the user. In some embodiments, the instruction from the caregiver computing device 512 is processed by the user computing device 510 based on a type of the caregiver.

As shown in FIG. 5, the pump 502 may be in communication with both the user computing device 510 (via a link 555) and the monitoring system 504 (via a link 551) according to some embodiments. In these embodiments, the monitoring system 504 transmits to the pump 502 signals indicating a blood glucose level of the user at each time step. The pump 502 may execute an algorithm for closed-loop operation based on the signals from the monitoring system 504 and additional parameters associated with the user from the user computing device 510.

In some embodiments, the pump 502 is in communication only with the user computing device 510 (via the link 555), which itself communicates with the monitoring system 504 (via a link 552). In these embodiments, either the pump 502 or the user computing device 510 may run the algorithm for medicament delivery. If the user computing device 510 manages the algorithm, it will send instructions (e.g., flow rate, bolus parameters) to the pump 502 for execution.

In some embodiments, the monitoring system 504 is in communication directly to the communication network 518 (via a link 553), such that any device connected to the communication network 518 can access the data uploaded by the monitoring system 504 to the server 520 via the communication network 518. That is, according to different embodiments, the monitoring system 504 can be in communication with the pump 502 via the link 551, with the user computing device 510 via the link 552, and/or with the communication network 518 via the link 553.

In some embodiments, at least one of the computing devices 510, 512, 514 is further operable to communicate with the database 516 over the communication network 518. For example, the user computing device 510 can store data to, and read data from, the database 516. The database 516 can be a remote storage device, such as a cloud-based server, a disk (e.g., a hard disk), a memory device on another application server, a networked computer, or any other suitable remote storage. Although shown remote to the user computing device 510, in some examples, the database 516 can be a local storage device, such as a hard drive, a non-volatile memory, or a USB stick. For example, the user computing device 510 may store the dosing information received from the pump 502 and/or from the monitoring system 504 in the database 516. The user computing device 510 may receive measurement data from the monitoring system 504 and store them in the database 516. The user computing device 510 may also store previous dosing history data and/or pump state history data received from the pump 502 in the database 516. In addition, the caregiver computing device 512 may store previous instruction data transmitted to the user computing device 510 in the database 516.

In some examples, the user computing device 510 generates and/or updates different models (e.g., online dosing model, offline dosing model, open loop model, etc.) for medicament dose computation. When some models are machine learning models or deep learning models, the user computing device 510 may generate training data for the models based on data including but not limited to: historical dosing information of the pump 502, historical dosing data of the pump 502, historical blood glucose levels of the user, historical pump states of the pump 502. The user computing device 510 trains the models based on their corresponding training data, and store the models in a database, such as in the database 516 (e.g., a cloud storage or a local storage). The models, when executed by the pump 502, allow the pump 502 to determine and deliver doses of medicament to the user. In some examples, the user computing device 510 assigns the models (or parts thereof) for execution to one or more processing devices (not shown) connected to the communication network 518. For example, each model may be assigned to a virtual machine hosted by a processing device. The virtual machine may cause the models or parts thereof to execute on one or more processing units such as GPUs. In some examples, the virtual machines assign each model (or part thereof) among a plurality of processing units. Based on the output of the models, the user computing device 510 and/or the pump 502 may determine medicament infusion data.

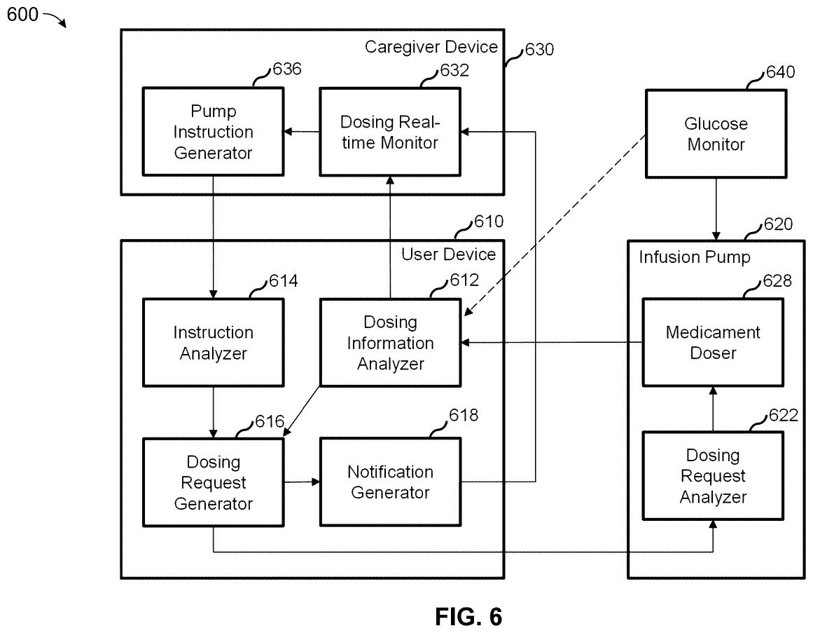

FIG. 6 is a block diagram of a system 600 for employing automated medicament delivery with a user device, a caregiver device and an infusion pump, according to various embodiments of the present disclosure. Referring to FIG. 6, the system 600 includes a user device 610, an infusion pump 620, a caregiver device 630 and a glucose monitor 640. In some embodiments, an automated glycemic control algorithm is implemented across the user device 610, the infusion pump 620 and/or the caregiver device 630 to provide optimized insulin delivery to a user.

In some embodiments, the user device 610 is a mobile device (e.g., a phone, tablet, watch, wearable device or laptop) associated with the user, the caregiver device 630 is another mobile device (e.g., a phone, tablet, watch, wearable device or laptop) associated with a caregiver of the user, and the infusion pump 620 is configured to deliver medicament doses to the user. In some examples, the user device 610 may be implemented as the computing device 510 shown in FIG. 5; and the caregiver device 630 may be implemented as the computing device 512 shown in FIG. 5. The infusion pump 620 may be implemented as the pump 12 shown in FIG. 1 and FIG. 2, the pump 102 shown in FIG. 3, or the pump 502 shown in FIG. 5. In some embodiments, each of the user device 610, the caregiver device 630 and the infusion pump 620 includes components connected in a scheme as shown in the housing 26 in FIG. 2. Both the caregiver device 630 and the infusion pump 620 are communicatively coupled to the user device 610.

As shown in FIG. 6, the user device 610 includes a dosing information analyzer 612, an instruction analyzer 614, a dosing request generator 616 and a notification generator 618. The infusion pump 620 in this example includes: a dosing request analyzer 622 and a medicament doser 628. The caregiver device 630 in this example includes: a dosing real-time monitor 632 and a pump instruction generator 636. In some examples, one or more of the dosing information analyzer 612, the instruction analyzer 614, the dosing request generator 616, the notification generator 618, the dosing request analyzer 622, the medicament doser 628, the dosing real-time monitor 632 and the pump instruction generator 636 are implemented in hardware. In some examples, one or more of the dosing information analyzer 612, the instruction analyzer 614, the dosing request generator 616, the notification generator 618, the dosing request analyzer 622, the medicament doser 628, the dosing real-time monitor 632 and the pump instruction generator 636 are implemented as an executable program maintained in a tangible, non-transitory memory, such as the memory 30 in FIG. 2, which may be executed by one or more processors, such as the processor 42 in FIG. 2.

The dosing information analyzer 612 in this example can receive dosing information associated with the user from the infusion pump 620. The dosing information may comprise at least one of: data from a CGM, e.g., the glucose monitor 640, associated with the user, an estimated blood-glucose level (BGL) of the user e.g., based on the data from the CGM, a dosing history of the infusion pump 620 regarding the user, or information about a current pump state of the infusion pump 620.

In some embodiments, the glucose monitor 640 is merely in communication with the infusion pump 620, without communicating to the user device 610. In these embodiments, the infusion pump 620 collects all of these dosing information, based on historical data generated by the infusion pump 620 and data received from the glucose monitor 640, and transmits the dosing information to the dosing information analyzer 612 of the user device 610. For example, the infusion pump 620 may generate the dosing information associated with the user based on filtered measurement data from the glucose monitor 640 and a pump state determined for the infusion pump 620. The infusion pump 620 in this example may filter measurement data of the user from the glucose monitor 640. In some embodiments, an estimated value is computed based on applying a filter on the measurement result on the user from the monitor 640.

In some embodiments, the glucose monitor 640 is in direct communication with the user device 610, without communication to the infusion pump 620. In these embodiments, the dosing information analyzer 612 collects the dosing information both from the infusion pump 620 and from the glucose monitor 640.

After obtaining the dosing information, the dosing information analyzer 612 may analyze the dosing information by extracting data from the dosing information, and forward the data to the dosing request generator 616 for dosing request generation. In some embodiments, the dosing information analyzer 612 also forwards the data in real-time to the dosing real-time monitor 632 of the caregiver device 630, such that the dosing real-time monitor 632 can monitor health data or dosing data of the user in real-time.

In some embodiments, the dosing real-time monitor 632 of the caregiver device 630 can analyze the dosing information received from the dosing information analyzer 612 and forward the analyzed dosing information to the pump instruction generator 636 for pump instruction generation. For example, the dosing real-time monitor 632 may determine an event based on the analysis of the dosing information, where the event may indicate that: the BGL of the user is above a certain threshold, the BGL of the user is above a threshold for longer than a certain time period, or a previous dose of medicament to the user is too much or too little based on the BGL of the user, etc. Once detecting this kind of event, the dosing real-time monitor 632 may trigger the pump instruction generator 636 to generate an instruction regarding the next bolus or future setting for the infusion pump 620, e.g., by sending the analyzed dosing information to the pump instruction generator 636.

In some embodiments, the dosing real-time monitor 632 may collect the dosing information from the glucose monitor 640, e.g., via a cloud network. In some embodiments, the dosing real-time monitor 632 may collect the dosing information from both the user device 610 and the glucose monitor 640, e.g., via one or more cloud networks. In some embodiments, all the data related to medicament delivery and available to the user device 610, the infusion pump 620 and the glucose monitor 640 are transmitted to the dosing real-time monitor 632 in real-time, directly or indirectly.

After obtaining the analyzed dosing information from the dosing real-time monitor 632, the pump instruction generator 636 may generate, based on the dosing information, an instruction with respect to medicament delivery from the infusion pump 620 to the user. The instruction may include a bolus request for a current dosing interval regarding the medicament delivery from the infusion pump 620 to the user, or include an update request for updating a configuration or setting of the infusion pump 620. The pump instruction generator 636 can transmit the instruction to the instruction analyzer 614 of the user device 610.

In some embodiments, the instruction is automatically generated by the pump instruction generator 636, in response to the monitored dosing information by the dosing real time monitor 632. For example, when the monitored dosing information satisfies a predetermined condition (e.g., when the BGL of the user is above a certain threshold, or when the dosing history of the infusion pump 620 indicates a continuous dosing for a time period longer than a certain threshold), the pump instruction generator 636 is automatically triggered to generate the instruction correspondingly. In some embodiments, the instruction is generated by the pump instruction generator 636, in response to an input from the caregiver. The input may be based on a visualization of the dosing information displayed on the caregiver device 630.

The instruction analyzer 614 of the user device 610 in this example obtains the instruction from the pump instruction generator 636 of the caregiver device 630. Based on an analysis of the instruction, the instruction analyzer 614 can determine a type of the instruction, and send the instruction along with its metadata indicating the instruction type to the dosing request generator 616.

In some examples, the instruction includes a first bolus request for a current dosing interval regarding the medicament delivery from the infusion pump 620 to the user. The first bolus request may be generated by the pump instruction generator 636 based on the analyzed dosing information associated with the user regarding a pervious or current dosing interval. In these examples, the dosing request generator 616 is configured to: generate a second bolus request for the current dosing interval regarding the medicament delivery based on the dosing information received from the dosing information analyzer 612, receive the first bolus request for the current dosing interval from the pump instruction generator 636, and determine a dosing request based on at least one of the first bolus request and the second bolus request.

In some embodiments, to determine the dosing request, the dosing request generator 616 first determines whether the caregiver device 630 is authorized to instruct a bolus request to the infusion pump 620. In accordance with a determination that the caregiver device 630 is not authorized to instruct a bolus request to the infusion pump 620, the dosing request generator 616 ignores the first bolus request when determining a bolus target for the dosing request. In accordance with a determination that the caregiver device 630 is authorized to instruct a bolus request to the infusion pump 620, the dosing request generator 616 then determines whether the user device 610 has already sent the second bolus request for the current dosing interval to the infusion pump 620.

In accordance with a determination that the user device 610 has already sent the second bolus request to the infusion pump 620, the dosing request generator 616 can compute a difference between the first bolus request and the second bolus request, and determine a bolus target for the dosing request based on the difference. For example, if the second bolus request was sent to request the infusion pump 620 to deliver a bolus size A, and the first bolus request indicates that the infusion pump 620 should deliver a bolus size B, the dosing request generator 616 may determine a bolus target for the dosing request, which will be sent to request the infusion pump 620 to deliver a bolus size B-A for the current dosing interval, if B is larger than A. If B is not larger than A, the dosing request generator 616 may either cancel the dosing request (as the additional bolus request for the current dosing interval), or generate the dosing request (as next bolus request for next dosing interval) with a reduction of bolus size by A-B.

In accordance with a determination that the user device 610 has not sent the second bolus request to the infusion pump 620, the dosing request generator 616 can compute a combined bolus request based on the first bolus request and the second bolus request, and determine a bolus target for the dosing request based on the combined bolus request.

In some examples, the combined bolus request is computed based on selecting one of the first bolus request and the second bolus request based on a predetermined policy. For example, if the user has previously authorized the caregiver hosting the caregiver device 630 as a trusted caregiver with delegation right, or if the user has previously identified the caregiver device 630 as a trusted caregiver device with delegation right, the dosing request generator 616 may select the first bolus request to generate the bolus target for the dosing request. If the first bolus request is not from a caregiver or caregiver device with delegation right, the dosing request generator 616 can select the second bolus request to generate the bolus target for the dosing request.

In some examples, the combined bolus request is computed based on computing a weighted combination of the first bolus request and the second bolus request based on weights previously assigned to the caregiver device 630 and the user device 610, respectively. For example, the user may have previously assigned a first weight of 25% to the caregiver device 630 and assigned a second weight of 75% to the user device 610, such that the combined bolus request is computed based on a summation of 25% of the bolus value of the first bolus request and 75% of the bolus value of the second bolus request. In some examples, the first weight is dynamically updated based on feedback data from the infusion pump 620 and/or the glucose monitor 640. For example, the first weight may be increased automatically after the feedback data indicates that historical bolus requests from the caregiver device 630 are more accurate than the bolus requests generated by the dosing request generator 616, or that the bolus requests from the caregiver device 630 gradually become more accurate than before. The second weight may be dynamically updated accordingly based on the updated first weight.

In some examples, the instruction includes an update request for updating a configuration of the infusion pump 620. The update request may be generated by the pump instruction generator 636 based on the analyzed dosing information associated with the user regarding historical dosing intervals. For example, the configuration may be related to a dosing function of the infusion pump 620, a reaction rate of the infusion pump 620 in response to a change of pump state, a length of each dosing interval for the infusion pump 620, a threshold of a BGL to trigger an alert or event for the user, or a maximum or minimum dosing size for each dosing interval, etc.

In some embodiments, the user may get a new pump, and want to swap the infusion pump 620 with the new pump. In accordance with the scheme of the system 600, the caregiver for the user can update settings on the user's new pump through the caregiver device 630, without requiring the user to make an in-person appointment with the caregiver.

In some examples, the dosing request generator 616 is configured to: receive the update request from the pump instruction generator 636, and determine whether the caregiver device 630 is authorized to instruct an update to the infusion pump 620. In accordance with a determination that the caregiver device 630 is not authorized to instruct an update to the infusion pump 620, the dosing request generator 616 may ignore the update request without changing the configuration of the infusion pump 620. In accordance with a determination that the caregiver device 630 is authorized to instruct an update to the infusion pump 620, the dosing request generator 616 may transmit the update request to the infusion pump 620 to update the configuration of the infusion pump 620.

The notification generator 618 in the user device 610 can obtain the results of dosing request generation from the dosing request generator 616, and generate, based on the results, a notification for the caregiver device 630. For example, the notification generator 618 can transmit the notification to the dosing real-time monitor 632 of the caregiver device 630 to notify the results of the dosing request generation, indicating whether and how the instruction received from the caregiver device 630 was executed.

In some examples, when the instruction includes a first bolus request for a current dosing interval regarding the medicament delivery from the infusion pump 620 to the user, the notification generator 618 can transmit, based on the dosing request generation results of the dosing request generator 616, a notification to the dosing real-time monitor 632 in response to the first bolus request. For example, the notification may indicate: an acknowledgement of the receipt of the first bolus request; whether the first bolus request was denied or granted; why the first bolus request was denied or granted; and if granted, how the first bolus request was executed or incorporated into the dosing request; etc.

In some examples, in accordance with a determination that the caregiver device 630 is not authorized to instruct a bolus request to the infusion pump 620, the notification indicates that the caregiver device 630 is not authorized to instruct a bolus request to the infusion pump 620, and that the first bolus request was thus denied. In accordance with a first determination that the caregiver device 630 is authorized to instruct a bolus request to the infusion pump 620 and a second determination that the user device 610 has already sent the second bolus request to the infusion pump 620, the notification indicates that: the first bolus request was granted, and the difference between the first bolus request and the second bolus request was instructed to the infusion pump 620 (e.g., for the current dosing interval or next dosing interval). In accordance with a third determination that the caregiver device 630 is authorized to instruct a bolus request to the infusion pump 620 and a fourth determination that the user device 610 has not sent the second bolus request to the infusion pump 620, the notification indicates that: the first bolus request was granted, and the combined bolus request (e.g., based on a weighted combination of the first bolus request and the second bolus request) was instructed to the infusion pump 620 for the current dosing interval.

In some examples, when the instruction includes an update request for updating a configuration of the infusion pump 620, the notification generator 618 can transmit, based on the dosing request generation results of the dosing request generator 616, a notification to the dosing real-time monitor 632 in response to the update request. For example, the notification may indicate: an acknowledgement of the receipt of the update request; whether the update request was denied or granted; why the update request was denied or granted; and if granted, when and how the update request will be executed by the infusion pump 620; etc.

In some examples, in accordance with a determination that the caregiver device 630 is not authorized to instruct an update to the infusion pump 620, the notification sent to the dosing real-time monitor 632 may indicate that the caregiver device 630 is not authorized to instruct an update to the infusion pump 620, and that the update request was thus denied. In accordance with a determination that the caregiver device 630 is authorized to instruct an update to the infusion pump 620, the notification sent to the dosing real-time monitor 632 may indicate that: the update request was granted, and the corresponding configuration of the infusion pump 620 is updated (e.g., by end of the current dosing interval, starting from the next dosing interval, or by a certain time in future).

In some embodiments, the notification sent to the dosing real-time monitor 632 is provided to the caregiver, e.g., via a user interface of the caregiver device 630. The notification may be a message on a screen or a vibration in a particular pattern. The dosing real-time monitor 632 may keep monitoring the notifications sent by the notification generator 618 and provide updated dosing information to the pump instruction generator 636 based on the notifications. The pump instruction generator 636 may generate and transmit new instructions using the updated dosing information including the previous notifications as feedback data.

In some embodiments, at least one of the user device 610 or the infusion pump 620 is configured to provide to the user, e.g., via a user interface of the user device 610 or the infusion pump 620, a notification that the medicament delivery of the infusion pump 620 was instructed by the caregiver device 630, or a notification that a configuration or setting of the infusion pump 620 was updated by the caregiver device 630. The notification may include at least one of: a message on a screen or a vibration in a particular pattern.

The dosing request analyzer 622 of the infusion pump 620 may obtain the dosing request from the dosing request generator 616 of the user device 610, and analyze the dosing request. In some embodiments, the dosing request is updated and transmitted from the dosing request generator 616 of the user device 610 to the dosing request analyzer 622 of the infusion pump 620 at periodic time intervals. The dosing request analyzer 622 may analyze the dosing request by extracting data from the dosing request, and forward the data to the medicament doser 628 for dosing execution. Based on the data extracted from the dosing request, the medicament doser 628 of the infusion pump 620 may determine a dose of medicament, and deliver the dose of medicament to the user.

In some embodiments, the dosing request received by the dosing request analyzer 622 includes a dosing model that indicates a plurality of dosing functions corresponding to a plurality of predicted pump states of the infusion pump 620 respectively. Each of the dosing functions represents a corresponding dosing target associated with a corresponding predicted pump state. The dosing model is customized to the user based on one or more characteristics of the user. The one or more user characteristics comprise at least one of: a target blood glucose, a basal rate, a correction factor, and a carb ratio.

In some embodiments, the dose of medicament is determined by the medicament doser 628 based on: determining a current pump state of the infusion pump 620; determining a predicted pump state of the dosing model that corresponds to the current pump state; and determining the dose of medicament based on the dosing model and a respective dosing function corresponding to the predicted pump state in the dosing model.

In some embodiments, a same software application, which supports both caregiver and patient accounts, is installed on the user device 610 and the caregiver device 630. In some examples, a patient account is signed in by the user via the software application on the user device 610.

In some embodiments, the caregiver device 630 may be authorized to receive real-time dosing information from the user device 610 and to send instructions to the user device 610, based on a two-step authorization process. When a caregiver account is signed in by the caregiver via the software application on the caregiver device 630, a first authorization is determined based on whether the caregiver account has been associated with the patient account according to a previous agreement from the user. Whenever an instruction is received from the caregiver device 630, a second authorization is determined based on whether the instruction is generated from a previously authorized caregiver account.

In some embodiments, the two-step authorization process is performed by the dosing request generator 616 of the user device 610. In some embodiments, the two-step authorization process is performed by the instruction analyzer 614 of the user device 610. In some embodiments, the two-step authorization process is performed by a server hosting a cloud network connecting the user device 610 and the caregiver device 630, e.g., the server 520 hosting the communication network 518 in FIG. 5. In some embodiments, after a device (e.g., the user device 610 or the caregiver device 630) is authorized, an access token may be received by the server 520 to gain access related capabilities on the device for medicament delivery.

In some embodiments, one or more of the components in one device of the system 600 can be shifted to another device of the system 600. For example, the dosing information analyzer 612 can be shifted from the user device 610 to the infusion pump 620. The function of the dosing request analyzer 622 can be shifted from the infusion pump 620 to the user device 610. In some embodiments, all computations for determining the optimal insulin dose are performed by the user device 610. In some embodiments, only computations having a complexity or intensity higher than a threshold for determining the optimal insulin dose are performed by the user device 610.

In some embodiments, the 614, the 616 and/or the 618 can be shifted from the user device 610 to the 630. This may happen when: the 610 is associated with a user lacking operating capacity, the caregiver hosting the 630 has previously authorized to take fully control of the 610 on behalf of the user, the 610 has a limited capacity due to an age or capacity limitation on the user, etc. For example, a parent of a child user may disable some functionalities of the 610 of the child user related to medicament delivery of the 620 to the child user, such that the dosing request and other related decisions are made by the 630 associated with the parent, rather than by the 610 associated with the child user.

In some embodiments, the infusion pump 620 itself can be the computation center and decision maker. For example, the infusion pump 620 may collect all dosing information based on data from the glucose monitor 640, and receive the instruction from the caregiver device 630 via the user device 610. The infusion pump 620 can determine the dosing target for each dosing interval based on the dosing information and the instruction, in a similar manner as what the dosing request generator 616 does as described above.

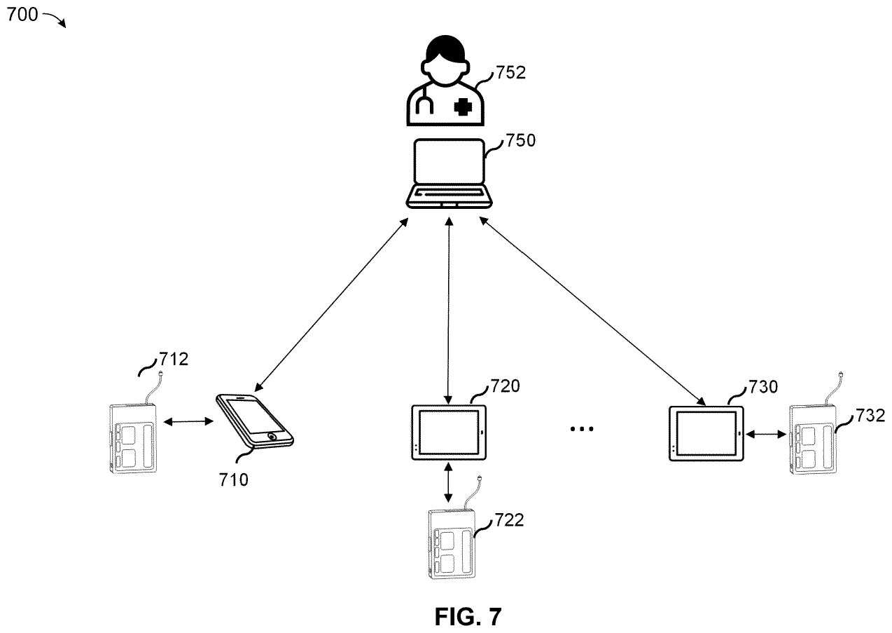

FIG. 7 illustrates a scenario 700 when one caregiver 752 is associated with multiple users for medicament delivery, according to various embodiments of the present disclosure. As shown in FIG. 7, the caregiver 752 is hosting a caregiver device 750, which is associated with multiple user devices 710, 720, 730. The multiple user devices 710, 720, 730 are configured to generate dosing requests for the infusion pumps 712, 722, 732, respectively. The scenario 700 may happen when a same caregiver is a doctor or nurse for multiple patients at the same time.

In some embodiments, a caregiver account is logged in by the caregiver 752 on the caregiver device 750. The caregiver account on the caregiver device 750 is authorized to instruct a plurality of patient accounts, each of which is logged in by a user on a respective one of the multiple user devices 710, 720, 730. That is, the caregiver device 750 may work in concert with each one of the multiple user devices 710, 720, 730, in a similar manner as how the caregiver device 630 works together with the user device 610 in FIG. 6. In some examples, when the caregiver device 750 transmits an instruction to one of the multiple user devices 710, 720, 730, e.g., the user device 710, the instruction includes at least one identification uniquely identifying the user device 710 or the patient account logged in on the user device 710.

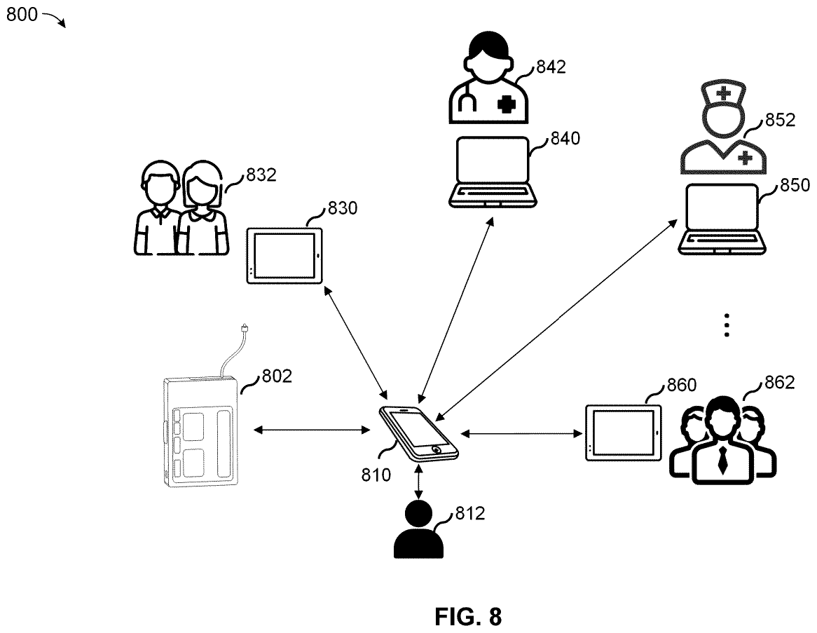

FIG. 8 illustrates a scenario 800 when one user is associated with multiple caregivers for medicament delivery to the user, according to various embodiments of the present disclosure. As shown in FIG. 8, a same user 812 may have different types of caregivers including: parents 832 of the user 812, healthcare professionals 842 for the user 812, school nurses 852 of the user 812, and responsible adults and/or legal representatives 862 of the user 812. The scenario 800 may happen when these caregivers are all responsible for a same patient at the same time, especially when the patient is a kid whose age is under a certain threshold or a person having no legal capacity or self-care capability, according to regulations, polices and/or laws in corresponding jurisdictions.

As shown in FIG. 8, the multiple caregivers 832, 842, 852, 862 are hosting caregiver devices 830, 840, 850, 860, respectively. Each of the caregiver devices 830, 840, 850, 860 is associated with the user device 810. The user device 810 is configured to generate dosing requests for the infusion pump 802.

In some examples, a patient account is logged in by the user 812 on the user device 810. The patient account on the user device 810 is configured to receive instructions from a plurality of authorized caregiver accounts, each of which is logged in by a respective caregiver of the caregivers 832, 842, 852, 862, on the caregiver devices 830, 840, 850, 860, respectively.

In some examples, each of the caregiver devices 830, 840, 850, 860 may work in concert with the user device 810, in a similar manner as how the caregiver device 630 works together with the user device 610 in FIG. 6. For example, when one of the caregiver devices 830, 840, 850, 860, e.g., the caregiver device 830, transmits an instruction to the user device 810, the instruction includes at least one identification uniquely identifying the user device 810 and/or the caregiver device 830. In some examples, the instruction from the caregiver device 830 is processed at the user device 810 based on a type of the caregiver 832 hosting the caregiver device 830.

In some embodiments, to add a new caregiver device associated with a new caregiver for a given user, the user can initiate an invitation process from the software application installed on a user device of the user. The invitation process may include sending a message, e.g., a text message or an email, to the new caregiver device, from the user device or from a cloud server connected to the user device. The message includes an invitation and instructions for the new caregiver to download and install the software application on the new caregiver device, create a caregiver account, and log in the caregiver account via the software application on the new caregiver device. The cloud server may link the newly created caregiver account to the patient account of the user, and record the newly created caregiver account as an authorized caregiver account for the user.

In some embodiments, one of the caregivers 832, 842, 852, 862 for the user 812 may take control of the user device 810. For example, the parents 832 of the user 812 may disable some capability of the user device 810, or shift some capability of the user device 810 related to medicament delivery to the caregiver device 830.

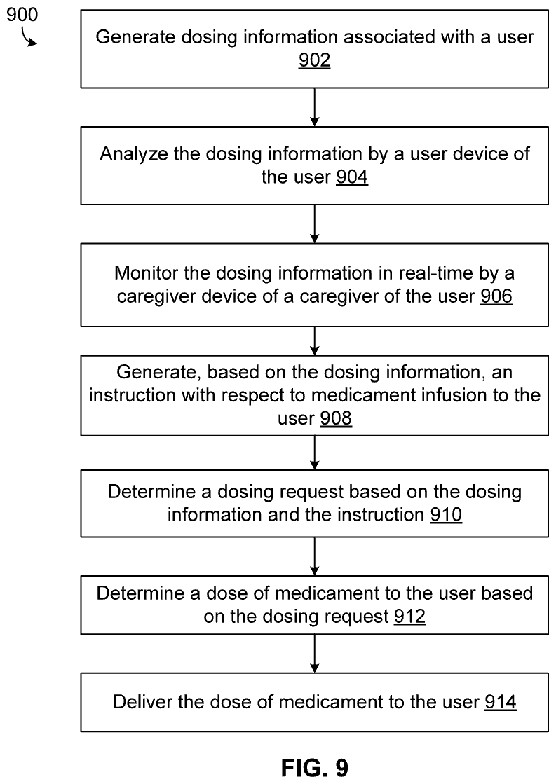

Referring now to FIG. 9, a flow chart of a method 900 for medicament delivery is depicted, according to various embodiments of the present disclosure. In some embodiments, the method 900 can be implemented through the system 600 in FIG. 6.

At step 902, dosing information associated with a user is generated. In some embodiments, the step 902 is performed by the infusion pump 620 and/or the glucose monitor 640. The dosing information comprises at least one of: measurement data from the glucose monitor 640 associated with the user, an estimated blood-glucose level (BGL) of the user based on the measurement data, a dosing history of the infusion pump 620 regarding the user, or information about a current pump state of the infusion pump 620.

The dosing information is analyzed at step 904 by a user device of the user, e.g., by the user device 610. In some embodiments, the step 904 is performed by the dosing information analyzer 612 in the user device 610.

At step 906, the dosing information is monitored in real-time by a caregiver device of a caregiver of the user, e.g., by the caregiver device 630. In some embodiments, the step 906 is performed by the dosing real-time monitor 632 in the caregiver device 630. The dosing information may be transmitted in real-time from the dosing information analyzer 612 to the dosing real-time monitor 632.

Based on the dosing information, an instruction is generated at step 908 with respect to medicament infusion to the user. In some embodiments, the step 908 is performed by the pump instruction generator 636 in the caregiver device 630. In some examples, the instruction includes a bolus request for a current dosing interval regarding the medicament delivery from the infusion pump 620 to the user. In some examples, the instruction includes an update request for updating a configuration or setting of the infusion pump 620.

A dosing request is determined at step 910, based on the dosing information and the instruction. In some embodiments, the step 910 is performed by the dosing request generator 616 in the user device 610. As discussed above, depending on the type of the instruction, the dosing request may include a bolus request based on a difference or a combination for medicament delivery from the infusion pump 620 to the user, or include the update request for updating a configuration or setting of the infusion pump 620.

At step 912, a dose of medicament to the user is determined based on the dosing request. In some embodiments, the step 912 is performed by the dosing request analyzer 622 in the infusion pump 620. The dosing request may be transmitted from the dosing request generator 616 to the dosing request analyzer 622. At step 914, the dose of medicament is delivered to the user, which may be performed by the medicament doser 628 in the infusion pump 620.

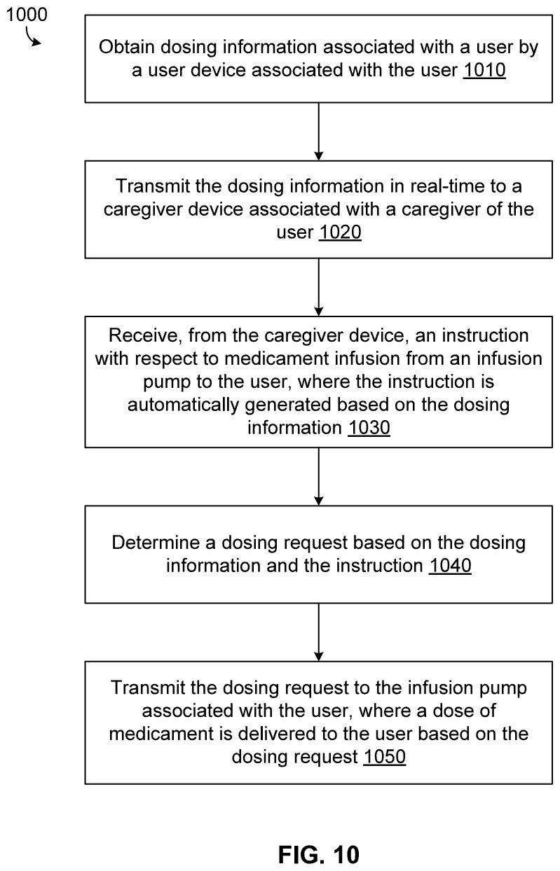

FIG. 10 is a flow chart of a method 1000 performed by a user device for delivering a dose of medicament, according to various embodiments of the present disclosure. In some embodiments, the method 1000 is performed by a user device associated with the user, e.g., the user computing device 510 shown in FIG. 5, or the user device 610 shown in FIG. 6.

In the illustrated embodiment, the user device obtains (at step 1010) dosing information associated with a user. The dosing information may include but not limited to: data from a CGM, e.g., the glucose monitor 640, associated with the user, an estimated blood-glucose level (BGL) of the user based on the data, a dosing history of an infusion pump, e.g., the pump 502 in FIG. 5 or the infusion pump 620 in FIG. 6, regarding the user, or information about a current pump state of the infusion pump. In some embodiments, the step 1010 is performed by the dosing information analyzer 612 in the user device 610.

Then, the user device transmits (at step 1020) the dosing information in real-time to a caregiver device associated with a caregiver of the user (e.g., the caregiver computing device 512 in FIG. 5 or the caregiver device 630 in FIG. 6). In some embodiments, the step 1020 is performed by the dosing information analyzer 612 in the user device 610.

At step 1030, the user device receives, from the caregiver device, an instruction with respect to medicament infusion from the infusion pump to the user. In some examples, the instruction is generated based on the dosing information by the caregiver device. In some embodiments, the step 1030 is performed by the instruction analyzer 614 in the user device 610. In some examples, the instruction analyzer 614 may analyze the instruction and send the analysis results to the dosing request generator 616 in the user device 610.

Based on the dosing information and the instruction, the user device determines (at step 1040) a dosing request. In some embodiments, the step 1040 is performed by the dosing request generator 616 in the user device 610. As discussed above, depending on the type of the instruction, the dosing request may include a bolus request based on a difference or a combination for medicament delivery from the infusion pump (e.g., the pump 502 in FIG. 5 or the infusion pump 620 in FIG. 6) to the user, or include an update request for updating a configuration or setting of the infusion pump.

At step 1050, the user device transmits the dosing request to the infusion pump associated with the user (e.g., the pump 502 in FIG. 5 or the infusion pump 620 in FIG. 6). In some embodiments, the step 1050 is performed by the dosing request generator 616 in the user device 610. A dose of medicament will be delivered by the infusion pump to the user based on the dosing request.

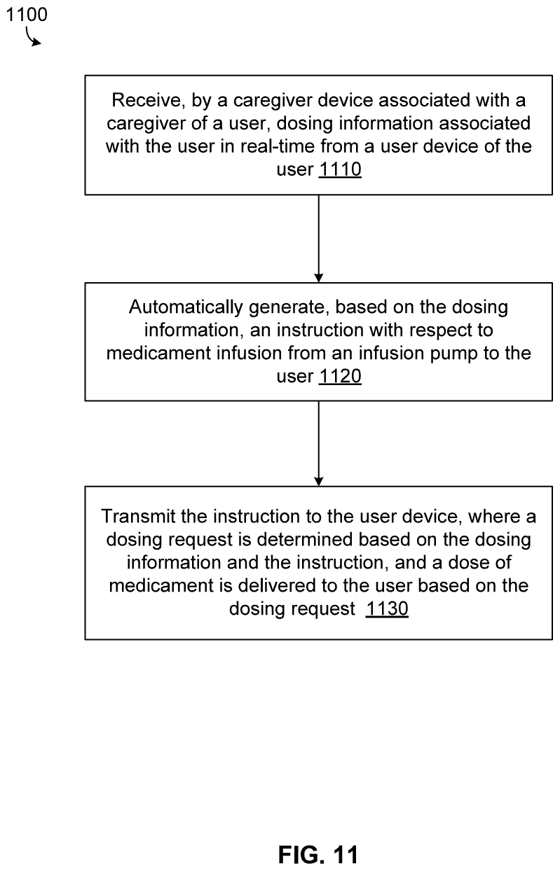

FIG. 11 is a flow chart of a method 1100 performed by a caregiver device for delivering a dose of medicament, according to various embodiments of the present disclosure. In some embodiments, the method 1100 is performed by a caregiver device associated with a caregiver of a user, e.g., the caregiver computing device 512 in FIG. 5 or the caregiver device 630 in FIG. 6.

In the illustrated embodiment, the caregiver device receives (at step 1110) dosing information associated with the user in real-time from a user device of the user, e.g., from the caregiver computing device 512 in FIG. 5 or the caregiver device 630 in FIG. 6. In some embodiments, the step 1110 is performed by the dosing real-time monitor 632 in the caregiver device 630. The dosing information may be generated by an infusion pump and/or a CGM associated with the user, and transmitted in real-time from a user device of the user, e.g., the pump 502 in FIG. 5 or the infusion pump 620 in FIG. 6.

After receiving the dosing information, the caregiver device generates (at step 1120), based on the dosing information, an instruction with respect to medicament infusion from the infusion pump to the user. In some embodiments, the step 1120 is performed by the pump instruction generator 636 in the caregiver device 630. As discussed above, the instruction may include a bolus request for a current dosing interval regarding the medicament delivery from the infusion pump to the user, or include an update request for updating a configuration or setting of the infusion pump.

At step 1130, the caregiver device transmits the instruction to the user device. In some embodiments, the step 1130 is performed by the pump instruction generator 636 in the caregiver device 630. A dosing request may be determined by the user device based on the dosing information and the instruction. A dose of medicament may be delivered by the infusion pump to the user based on the dosing request.

It should be understood that the individual operations used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

In various embodiments of the present disclosure, computing and other devices discussed herein can include memory. Memory can comprise volatile or non-volatile memory as required by the coupled computing device or processor to not only provide space to execute the instructions or algorithms, but to provide the space to store the instructions themselves. In one embodiment, volatile memory can include random access memory (RAM), dynamic random access memory (DRAM), or static random access memory (SRAM), for example. In one embodiment, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of the disclosure.

In one embodiment, the system or components thereof can comprise or include various modules or engines, each of which is constructed, programmed, configured, or otherwise adapted to autonomously carry out a function or set of functions. The term “engine” as used herein is defined as a real-world device, component, or arrangement of components implemented using hardware, such as by an application specific integrated circuit (ASIC) or field programmable gate array (FPGA), for example, or as a combination of hardware and software, such as by a microprocessor system and a set of program instructions that adapt the engine to implement the particular functionality, which (while being executed) transform the microprocessor system into a special-purpose device.

An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations and should generally not be limited to any particular implementation exemplified herein, unless such limitations are expressly called out. In addition, an engine can itself be composed of more than one sub-engine, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. For example, in an embodiment, each of the processes depicted in FIG. 5 could be implemented within engines as described above. Likewise, in other contemplated embodiments, multiple defined functionalities may be implemented by a single engine that performs those multiple functions, possibly alongside other functions, or distributed differently among a set of engines than specifically illustrated in the examples herein.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that embodiments may comprise fewer features than illustrated in any individual embodiment described above. The embodiments described herein are not meant to be an exhaustive presentation of the ways in which the various features may be combined. Accordingly, the embodiments are not mutually exclusive combinations of features; rather, embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Moreover, reference in the specification to “one embodiment,” “an embodiment,” or “some embodiments” means that a particular feature, structure, or characteristic, described in connection with the embodiment, is included in at least one embodiment of the teaching. The appearances of the phrase “in one embodiment” in various places in the specification are not necessarily all referring to the same embodiment.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein. For purposes of interpreting the claims, it is expressly intended that the provisions of Section 112, sixth paragraph of 35 U.S.C. are not to be invoked unless the specific terms “means for” or “step for” are recited in a claim.

1. (canceled)

2. A system for medicament delivery, the system comprising:
a caregiver device associated with a caregiver of a user, wherein the caregiver device is configured to:
obtain dosing information associated with the user, and
generate, based on the dosing information, an instruction with respect to medicament delivery to the user;
a user device associated with the user, wherein the user device is communicatively coupled to the caregiver device and configured to:
obtain the instruction from the caregiver device, and
determine a dosing request based on the instruction; and
an infusion pump associated with the user, wherein the infusion pump is communicatively coupled to the user device and configured to:
obtain the dosing request from the user device, and
deliver a dose of medicament to the user based on the dosing request.

3. The system of claim 2, wherein the dosing information comprises at least one of:
data from a continuous glucose monitor (CGM) associated with the user;
an estimated blood-glucose level (BGL) of the user;
a dosing history of the infusion pump regarding the user; or
information about a current pump state of the infusion pump.

4. The system of claim 2, wherein:
the instruction includes a first bolus request for a current dosing interval regarding the medicament delivery from the infusion pump to the user; and
the user device is further configured to:
generate a second bolus request for the current dosing interval regarding the medicament delivery based on the dosing information,
receive the first bolus request for the current dosing interval from the caregiver device,
determine the dosing request based on at least one of the first bolus request and the second bolus request, and
transmit, based on the dosing request, a notification to the caregiver device in response to the first bolus request.

5. The system of claim 4, wherein the dosing request is determined based on:
determining whether the caregiver device is authorized to instruct a bolus request to the infusion pump;
in accordance with a determination that the caregiver device is not authorized, ignoring the first bolus request when determining a bolus target for the dosing request;
in accordance with a determination that the caregiver device is authorized, determining whether the user device has already sent the second bolus request for the current dosing interval to the infusion pump;
in accordance with a determination that the user device has already sent the second bolus request to the infusion pump, computing a difference between the first bolus request and the second bolus request, and determining a bolus target for the dosing request based on the difference; and
in accordance with a determination that the user device has not sent the second bolus request to the infusion pump, computing a combined bolus request based on the first bolus request and the second bolus request, and determining a bolus target for the dosing request based on the combined bolus request.

6. The system of claim 5, wherein the combined bolus request is computed based on at least one of:
selecting one of the first bolus request and the second bolus request based on a predetermined policy; or
computing a weighted combination of the first bolus request and the second bolus request based on weights previously assigned to the caregiver device and the user device, respectively.

7. The system of claim 5, wherein:
in accordance with a determination that the caregiver device is not authorized, the notification indicates that the caregiver device is not authorized;
in accordance with a determination that the caregiver device is authorized and a determination that the user device has already sent the second bolus request to the infusion pump, the notification indicates that the difference between the first bolus request and the second bolus request is instructed to the infusion pump; and
in accordance with a determination that the caregiver device is authorized and a determination that the user device has not sent the second bolus request to the infusion pump, the notification indicates that the combined bolus request is instructed to the infusion pump.

8. The system of claim 2, wherein:
the instruction includes an update request for updating a configuration of the infusion pump; and
the user device is further configured to:
receive the update request from the caregiver device,
determine whether the caregiver device is authorized to instruct an update to the infusion pump,
in accordance with a determination that the caregiver device is not authorized, ignore the update request and transmit a notification to the caregiver device to indicate that the caregiver device is not authorized, and
in accordance with a determination that the caregiver device is authorized, transmit the update request to the infusion pump to update the configuration of the infusion pump, and transmit a notification to the caregiver device to indicate that the configuration is updated.

9. The system of claim 2, wherein:
a software application is installed on the user device and the caregiver device;
a patient account is signed in by the user via the software application on the user device; and
the caregiver device is authorized based on:
when a caregiver account is signed in by the caregiver via the software application on the caregiver device, determining a first authorization based on whether the caregiver account has been associated with the patient account according to a previous agreement from the user; and
when the instruction is received from the caregiver device, determining a second authorization based on whether the instruction is generated from a previously authorized caregiver account.

10. The system of claim 9, wherein:
the caregiver account on the caregiver device is authorized to instruct a plurality of patient accounts, including the patient account on the user device; and
the instruction includes at least one identification uniquely identifying the patient account among the plurality of patient accounts.

11. The system of claim 9, wherein:
the patient account on the user device is configured to receive instructions from a plurality of authorized caregiver accounts, including the caregiver account on the caregiver device;
the authorized caregiver accounts are associated with different types of caregivers including: parents of the user, healthcare professionals for the user, school nurses of the user, responsible adults of the user, legal representatives of the user; and
the instruction from the caregiver is processed at the user device based on a type of the caregiver.

12. (canceled)

13. The system of claim 2, wherein:
the dosing request includes a dosing model that indicates a plurality of dosing functions corresponding to a plurality of predicted pump states respectively;
each of the plurality of dosing functions represents a corresponding dosing target associated with a corresponding predicted pump state;
the dosing model is customized to the user based on one or more characteristics of the user;
the one or more characteristics of the user comprise a target blood glucose, a basal rate, a correction factor, and a carb ratio; and
the dose of medicament is determined based on:
determining a current pump state of the infusion pump,
determining a predicted pump state of the dosing model that corresponds to the current pump state, and
determining the dose of medicament based on the dosing model and a respective dosing function corresponding to the predicted pump state in the dosing model.

14. An infusion pump comprising a processor and a non-transitory, computer-readable medium storing instructions which, when executed by the processor, cause the infusion pump to:
obtain a dosing request from a user device associated with a user, wherein:
the dosing request is determined based on an instruction with respect to medicament delivery from the infusion pump to the user,
the instruction is generated by a caregiver device associated with a caregiver of the user based on dosing information associated with the user, and
the dosing information is received in real-time by the caregiver device;
determine a dose of medicament for the user based on the dosing request; and
deliver the dose of medicament to the user.

15. The infusion pump of claim 14, wherein the dosing information comprises at least one of:
data from a continuous glucose monitor (CGM) associated with the user;
an estimated blood-glucose level (BGL) of the user;
a dosing history of the infusion pump regarding the user; or
information about a current pump state of the infusion pump.

16. The infusion pump of claim 14, wherein:
the instruction includes a first bolus request for a current dosing interval regarding the medicament delivery from the infusion pump to the user;
a second bolus request is generated by the user device for the current dosing interval regarding the medicament delivery based on the dosing information;
the dosing request is determined based on at least one of the first bolus request and the second bolus request according to a predetermined policy; and
a notification is transmitted, based on the dosing request, to the caregiver device in response to the first bolus request.

17. The infusion pump of claim 14, wherein:
the instruction includes an update request for updating a configuration of the infusion pump;
in accordance with a determination that the caregiver device is not authorized to instruct an update to the infusion pump, the update request is ignored and a notification is transmitted to the caregiver device to indicate that the caregiver device is not authorized; and
in accordance with a determination that the caregiver device is authorized to instruct an update to the infusion pump, the update request is transmitted to the infusion pump to update the configuration of the infusion pump, and a notification is transmitted to the caregiver device to indicate that the configuration is updated.

18. (canceled)

19. A user device comprising a processor and a non-transitory, computer-readable medium storing instructions which, when executed by the processor, cause the user device to:
receive an instruction from a caregiver device associated with a caregiver of a user, wherein:
the user device is associated with the user,
the instruction is with respect to medicament delivery from an infusion pump to the user,
the instruction is generated by the caregiver device based on dosing information associated with the user, and
the dosing information is received in real-time by the caregiver device;
determine a dosing request based on the instruction; and
transmit the dosing request to the infusion pump associated with the user, wherein a dose of medicament is delivered to the user based on the dosing request.

20. The user device of claim 19, wherein the user device is configured to:
determine that the instruction includes a first bolus request for a current dosing interval regarding the medicament delivery from the infusion pump to the user;
generate a second bolus request for the current dosing interval regarding the medicament delivery based on the dosing information;
determine the dosing request based on at least one of the first bolus request and the second bolus request; and
transmit, based on the dosing request, a notification to the caregiver device in response to the first bolus request.

21. The user device of claim 20, wherein the dosing request is determined based on:
determining whether the caregiver device is authorized to instruct a bolus request to the infusion pump;
in accordance with a determination that the caregiver device is not authorized, ignoring the first bolus request when determining a bolus target for the dosing request;
in accordance with a determination that the caregiver device is authorized, determining whether the user device has already sent the second bolus request for the current dosing interval to the infusion pump;
in accordance with a determination that the user device has already sent the second bolus request to the infusion pump, computing a difference between the first bolus request and the second bolus request, and determining a bolus target for the dosing request based on the difference; and
in accordance with a determination that the user device has not sent the second bolus request to the infusion pump, computing a combined bolus request based on the first bolus request and the second bolus request, and determining a bolus target for the dosing request based on the combined bolus request.

22. The user device of claim 21, wherein the combined bolus request is computed based on at least one of:
selecting one of the first bolus request and the second bolus request based on a predetermined policy; or
computing a weighted combination of the first bolus request and the second bolus request based on weights previously assigned to the caregiver device and the user device, respectively.

23. The user device of claim 21, wherein:
in accordance with a determination that the caregiver device is not authorized, the notification indicates that the caregiver device is not authorized;
in accordance with a determination that the caregiver device is authorized and a determination that the user device has already sent the second bolus request to the infusion pump, the notification indicates that the difference between the first bolus request and the second bolus request is instructed to the infusion pump; and
in accordance with a determination that the caregiver device is authorized and a determination that the user device has not sent the second bolus request to the infusion pump, the notification indicates that the combined bolus request is instructed to the infusion pump.

24-32. (canceled)
